(19) United States
(12) Patent Application Publication
(10) Pub. No.: US 2003/0044822 A1
(43) Pub. Date: Mar. 6, 2003

(54) Colorimetric in situ hybridization detection methods

(75) Inventors: Jonathan Fletcher, Brookline, MA (US); Sheng Xiao, Boston, MA (US)

Correspondence Address: Helen C. Lockhart, Wolf, Greenfield & Sacks, P.C., Federal Reserve Plaza, 600 Atlantic Avenue, Boston, MA, 02210, US

(73) Assignee: The Brigham and Women's Hospital, Inc., Boston, MA (US)

(21) Appl. No.: 10/132,993

(22) Filed: Apr. 26, 2002

Related U.S. Application Data
(62) Division of application No. 09/431,791, filed on Nov. 2, 1999.
(60) Provisional application No. 60/106,701, filed on Nov. 2, 1998.

(51) Int. Cl. C12Q 1/68; C12M 1/34

(52) U.S. Cl. 435/6; 435/287.2

(57) ABSTRACT

The invention is a method for genomic subtractive hybridization. Specific nucleic acid sequences are removed from a sample of nucleic acid sequences by specifically hybridizing the sequences to a complementary nucleic acid sequence bound to a target molecule such as biotin. The target molecule is then contacted with a binding partner such as avidin and separated from the sample of nucleic acid sequences. As the target is separated from the sample the hybridized nucleic acid sequences are also removed from the sample. The method preferably involves the removal of repetitive nucleic acid sequences from a nucleic acid sample to generate a library of probes that are substantially free of repetitive nucleic acid sequences.

### FIELD OF THE INVENTION

The present invention relates to a method for genomic subtractive hybridization. The invention also relates to methods for generating probes for in situ hybridization techniques. In particular, the methods for genomic hybridization are useful for generating probes for techniques such as in situ hybridization.

### BACKGROUND OF THE INVENTION

Chromosomes within a living cell encompass all of the DNA of a particular organism. The number and structure of chromosomes within a cell are indicative of various normal and abnormal developmental traits. For instance, the presence of a particular chromosome type or number of a particular chromosome may be indicative of an abnormal condition. Humans having three copies of chromosome 18 (trisomy) develop a fatal disorder causing death within one year of birth and subjects having three copies of chromosome 21 (trisomy 21) develop Downs syndrome. In general chromosome abnormalities can result from extra or missing individual chromosomes, extra or missing portions of a chromosome, or chromosomal rearrangements, including translocations, deletions, and inversions. The trisomies discussed above involve addition of chromosomal material. A translocation involves the transfer of a piece of a chromosome to another chromosome. An example of a disorder involving an exchange of chromosomal material is chronic myelogenous leukemia which involves a translocation of chromosomal material from chromosome 9 to chromosome 22. An inversion involves a reversal in polarity of a chromosomal segment. Dicentrics produce a chromosome with two centromeres.

Characteristic chromosome abberations have been described in a wide range of tumors. Specific oncogene and tumor suppressor gene targets affected by these chromosomal abnormalties have been characterized in some tumors but most of them remain to be studied. A major goal in studying human cancer chromosome and gene aberrations is to elucidate biological pathways responsible for neoplastic transformation. Several such pathways have already been identified through characterization of particular cancer chromosome aberrations (1, 3). Another goal in cancer chromosome evaluation is the identification and validation of novel diagnostic and prognostic markers (4, 5). Cytogenetic markers have utility as histological adjuncts, and methods that delineate diagnostic chromosomal aberrations or associated molecular changes are becoming increasingly important in experimental pathology (6-8).

Chromosomal abnormalities are generally detected in biological samples using standard methods for analyzing karyotypes. A karyotype defines the number and morphology of chromosomes in an individual or a related group of individuals. The number can be determined as the total chromosome number or the copy number of individual chromosome types. Chromosomal morphology can be determined for example, by measuring length of the chromosome or the centromeric index of the chromosome. Typically, techniques based on chemical staining have been used to produce bands on the chromosome that allow for the identification of each chromosome type. The banding technique has been used for years to identify chromosomal abnormalities such as translocations and inversions. (Latt, Optical Studies of Metaphase Chromosome Organization, Annual Review of Biophysics and Bioengineering, v. 5, p.1-37 (1976)).

The analysis of human tumors by the banding technique, however, has been difficult because primary human tumor cells generally are difficult to culture and there are often too few cells in metaphase to accurately band metaphase chromosomes. In order to accomplish the banding, the cells generally need to be stimulated to undergo cellular proliferation using mitogens. Tumor cells in a solid tumor often cannot even be stimulated to undergo cellular proliferation.

In situ hybridization (ISH) is an analytic method that enables evaluation of numerical and structural chromosome aberrations in both fresh and archival tumor and other tissue specimens (9-13) and has extended the capabilities of conventional tumor and other tissue karyotyping (14-17). ISH analysis using chromosome specific nucleic acid probes solve the problems associated with karyotyping of tumor cells with the banding technique by allowing the analysis of interphase nuclei, and thus avoiding the need to prepare metaphase chromosomes. Several advantages of ISH include applicability to interphase cell populations and the ability to demonstrate unambiguously chromosome rearrangements that target specific loci of interest (19-20). Several technical innovations have permitted the application of ISH in genome-wide screening studies, including comparative genomic hybridization (CGH) (21), combinatorial multifluor ISH (22), and multicolor spectral karyotyping (23). Each of these methods provides a broad perspective complementary to that provided by chromosome banding methods.

ISH has been used to identify the location of individual genes or other well defined nucleic acid sequences on chromosomes. Single copies of unique sequence probes have been used to identify the location of a particular gene on a chromosome. High background levels, however, often prevent reliable detection of small target sites.

Chromosome specific libraries have also been developed to generate probes useful for ISH to detect chromosome specific abnormalities. One method for generating chromosome specific libraries involves the use of flow cytometry to isolate a pure preparation of a chromosome from a pool of many individual chromosomes. The isolated chromosome may then be amplified by polymerase chain reaction (PCR) to manufacture a library (Chang et al., Genomics, v. 12, p. 307-312, 1992 and Boschman et al., Genes, Chromosomes, and Cancer, v. 6, p. 10-16, 1993). Additionally, chromosome specific libraries have been prepared from somatic cell hybrids (e.g., a rat-human hybrid cell line) which contains a few distinct human chromosomes. The chromosomes can be isolated and generated into a library without the requirement of a flow cytometry step. More recently, these types of libraries have been produced from subchromosomal regions using yeast, artificial chromosomes (YACs) that correspond to a region of interest on a particular chromosome (Lengauer et al., Hum. Mol. Genet., v. 2, p. 505-512, 1993).

### SUMMARY OF THE INVENTION

The present invention relates to methods and products for genomic subtractive hybridization. The methods are useful in particular for developing unique-sequence genomic probes suitable for ISH procedures. As described above, ISH is commonly used for pathological screening purposes to detect chromosomal defects. The genomic subtractive hybridization methods of the invention produce stable probes for ISH depleted of non-specific repeat sequences and thus produce less background than traditional methods of ISH. The probes produced according to these methods also are a stable resource of ISH DNA that can be labeled by many different types of methods including PCR, random priming or nick translation.

In one aspect the invention is a method for genomic subtractive hybridization. The method involves the steps of hybridizing a chemically modified oligonucleotide probe to a complementary nucleic acid sequence in a nucleic acid sample, wherein the chemically modified oligonucleotide probe is an oligonucleotide associated with a target molecule, and selectively removing the chemically modified oligonucleotide probe and complementary nucleic acid by selectively contacting the target molecule with a binding partner to produce binding partner/target conjugates and separating the binding partner and binding partner/target conjugates from the nucleic acid sample.

In one embodiment the target molecule is selected from the group consisting of avidin, biotin, fluorescein isothiocyanate (FITC), anti-FITC, antigen, and antibodies. Preferably the target molecule is biotin or FITC. In one embodiment the binding partner is immobilized on a support. In another embodiment the support is a bead and wherein the binding partner/target conjugates are separated from the nucleic acid sample by chromatography.

The oligonucleotide may have any specific nucleic acid sequence but preferably the oligonucleotide is a repetitive nucleic acid. In one embodiment the repetitive nucleic acid is isolated from YAC DNA or BAC DNA. In a preferred embodiment the chemically modified oligonucleotide probe is prepared by amplifying the repetitive nucleic acid using PCR and a primer attached to the target molecule.

The nucleic acid sample is a genomic nucleic acid sample in another embodiment. Preferably the genomic nucleic acid sample is obtained from a YAC, BAC, PAC, or P1 clone.

According to another aspect of the invention a library of nucleic acid probes for use in in situ hybridization methods is provided. The library includes a heterogenous mixture of labeled nucleic acid probes that are substantially complementary to unique nucleic acid fragments and are substantially free of repetitive nucleic acid sequences, and which are produced by the process of: (a) obtaining genomic nucleic acid fragments; (b) amplifying the genomic nucleic acid fragments; (c) hybridizing a chemically modified oligonucleotide probe to a complementary nucleic acid sequence in the genomic nucleic acid fragments, wherein the chemically modified oligonucleotide probe is an oligonucleotide associated with a target molecule; (d) selectively removing the chemically modified oligonucleotide probe and complementary nucleic acid by selectively contacting the target molecule with a binding partner and separating the binding partner and binding partner/target conjugates from the genomic nucleic acid fragments; and (e) labeling the genomic nucleic acid fragments with a label. Preferably the genomic nucleic acid fragments are labeled with biotin, digoxigenin, or a fluorescent molecule.

In one embodiment the heterogenous mixture of labeled nucleic acid probes are complementary to substantially all of the unique nucleic acid fragments in a genomic DNA population. In another embodiment the heterogenous mixture of labeled nucleic acid probes are complementary to substantially all of the unique nucleic acid fragments in a chromosome of a genomic DNA population. In yet another embodiment the heterogenous mixture of labeled nucleic acid probes are complementary to substantially all of the unique nucleic acid fragments in a subregion of a chromosome of a genomic DNA population.

In another aspect the invention is a method for performing in situ hybridization. The method includes the steps of hybridizing the library of labeled probes of the invention to a biological sample fixed on a surface, removing un-hybridized probe, and detecting a signal from the hybridized probe to identify nucleic acid sequences present in the biological sample.

According to another aspect of the invention a mixture of probes for genomic subtractive hybridization is provided. The probes of the mixture are isolated chemically modified oligonucleotide probes, wherein the chemically modified oligonucleotide probes are oligonucleotides associated with a target molecule selected from the group consisting of biotin, FITC, and wherein the oligonucleotide probes are a mixture of oligonucleotides complementary to repetitive nucleic acid sequences and yeast nucleic acid sequences. Preferably the target molecule is biotin.

In one embodiment the repetitive nucleic acid is isolated from YAC, BAC, PAC, or P1 clone. In another embodiment the chemically modified oligonucleotide probe is prepared by amplifying the repetitive nucleic acid using PCR and a primer attached to the target molecule.

Each of the limitations of the invention can encompass various embodiments of the invention. It is, therefore, anticipated that each of the limitations of the invention involving any one element or combinations of elements can be included in each aspect of the invention.

### BRIEF DESCRIPTION OF THE DRAWINGS

FIG. 1 is a chromosomal map depicting regions of chromosome 8 and chromosome 22.

FIG. 2 is a dot blot of subtracted and unsubtracted nucleic acid probes probed with Cot-1 (panel A) or total yeast genomic DNA (Panel B).

| | |
|---|---|
| Brief Description Of The Sequences | |
| SEQ. ID. NO.1 is a primer having the sequence 5′CTGAGCGGAATTCGTGAGACC | (T-1) |
| SEQ. ID. NO.2 is a primer having the sequence 5′GGTCTCACGAATTCCGCTCAGTT | (T-2) |
| SEQ. ID. NO.3 is a primer having the sequence 5′AATTCTTGCGCCTTAAACCAAC | (D-40) |
| SEQ. ID. NO.4 is a primer having the sequence 5′GTTGGTTTAAGGCGCAAG | (D-41). |
| SEQ. ID. NO.5 is a primer having the sequence 5′AATTCTTGCGCCTTAAACCAAC | (D-40B). |

### DETAILED DESCRIPTION OF THE INVENTION

The invention relates to a novel approach for expanding the role of in situ hybridization in diagnostic and research applications. Repetitive DNA sequences have been used in ISH to characterize various human chromosomes (Devillee et al., Cytogenet. Cell Genet., v. 41, p. 193-201 1986), for instance, to detect the presence of multiple loci on a chromosome. ISH probes derived from pericentromeric α-satellite repeat sequences have been used extensively to evaluate numerical chromosome aberrations. Many molecular cytogenetic research and diagnostic questions, however, are best addressed using unique sequence probes to particular chromosome regions. Large-insert clones, e.g., BAC, PAC, P1, and YAC clones, are suitable for molecular cytogenetic applications, including those using paraffin-embedded material. The fluorescence signals obtained using large-insert clones are often as bright as those obtained with α-satellite probes, particularly when the human genomic inserts are rich in unique sequences. The invention provides new methods and related products for preparing libraries of probes that are rich in unique sequences.

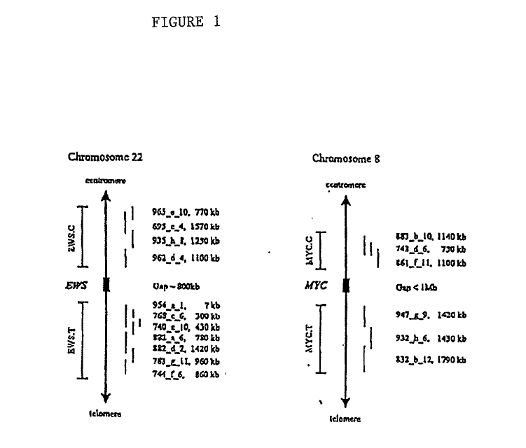

When used with the genomic subtractive hybridization methods of the invention these large probes are also suitable for bright-field studies, using either peroxidase/alkaline phosphatase, or sequential peroxidase detection strategies. In addition to the potential for excellent ISH signal intensity, a major advantage of mega-YACs is the wealth of physical mapping data accessible via the Internet at websites such as those of the Whitehead Institute and CEPH. These data facilitate selection of mega-YAC clones in a region of interest. As described in the Examples below multiple mega-YACs in the EWS and MYC regions have been tested and clones were selected with superior ISH signals for inclusion in the final contigs (FIG. 1). The probes (YAC contigs spanning 1.5 to 5.0 megabases) were treated by the subtractive hybridization methods of the invention to produce unique-sequence rich DNA.

The genomic subtractive hybridization approach of the invention accomplishes at least two objectives. The first is the removal of repetitive-sequences, and the second is the creation of a library of DNA fragments that can be manipulated readily for large-scale DNA preparation and labeling. Repetitive sequences in prior art ISH probes are generally competed through pre-annealing with total genomic or repeat-sequence enriched DNA.^{11,56} Pre-annealing can be an effective approach, but this step is both expensive and time consuming. In addition, some labeled repetitive sequences will likely come in contact with the slide. It is fundamentally desirable to remove these sequences from the probe altogether, thus eliminating a potential source of nonspecific fluorescence. The methods of the invention avoid these problems and thus accomplish the above-stated objectives.

In one aspect the invention is a method for genomic subtractive hybridization. The method involves the steps of hybridizing a chemically modified oligonucleotide probe to a complementary nucleic acid sequence in a nucleic acid sample, wherein the chemically modified oligonucleotide probe is an oligonucleotide associated with a target molecule, and selectively removing the chemically modified oligonucleotide probe and complementary nucleic acid by selectively contacting the target molecule with a binding partner to produce binding partner/target conjugates and separating the binding partner and binding partner/target conjugates from the nucleic acid sample.

A “chemically modified oligonucleotide probe” is an oligonucleotide associated with a target molecule. The oligonucleotide can have any nucleic acid sequence that is desirable. The purpose of the oligonucleotide is to hybridize to and remove a complementary nucleic acid from a mixture of nucleic acid sequences. Therefore it is desirable to have an oligonucleotide which has a nucleic acid sequence that is complementary to a nucleic acid sequence that will be removed. For instance when hybrid cells are used to generate chromosomes it is desirable to remove all non-human chromosomes. In this case the oligonucleotide would have a sequence complementary to the non-human sequences, such as yeast DNA. Preferably the oligonucleotide is a repetitive nucleic acid sequence (or a mixture of repetitive nucleic aid sequences and yeast DNA) which can be used to remove human repetitive sequences (repetitive sequences and yeast) from a library of oligonucleotide probes to reduce background levels in an ISH procedure. The oligonucleotide may be modified or unmodified. For instance the oligonucleotide may have a partial or complete phosphorothioate backbone.

As used herein “repetitive nucleic acid sequences” are nucleic acid sequences within a genome which encompass a series of nucleotides which are repeated many times, often in tandem arrays. The repetitive sequences can occur in the genome in multiple copies ranging from two to hundreds of thousands of copies and may be clustered or interspersed on one or more chromosomes throughout a genome. Repetitive nucleic acid sequence fragments when present in a pool of nucleic acid fragments which are denatured and then rehybridized, will rehybridize at a more rapid rate than the unique sequences. Unique sequences are those sequences having at least 12 nucleotides and which are present only once within an entire genome. Although the repetitive nucleic acid sequences are present throughout the genome a large number of the repetitive nucleic acid sequences are located at the centromere of each chromosome.

The human genome includes several families of repetitive nucleic acid sequences. For instance, alpha satellite nucleic acid sequences have variable numbers of tandem sequences of approximately 170 base pairs long. Satellite 2 nucleic sequences have repeat units of approximately 26 base pairs long. The repetitive nucleic acid sequences within a family are to highly homologous. These families also include subfamilies of repetitive sequences which have been produced by substitutions, deletions, and/or insertions or repetitive nucleic acid sequences. The degree of variation depends on the particular subfamily. As used herein the repetitive nucleic acid sequence refers to families and subfamilies which are capable of hybridizing with the dominate form of the family under normal stringency conditions of temperature, concentration, and time.

The oligonucleotide of the chemically modified oligonucleotide probe is associated with a target molecule. As used herein the term “associated” refers to a covalent interaction between the target molecule and the oligonucleotide.

The genomic nucleic acid fragments are hybridized with a chemically modified oligonucleotide probe which is complementary to some of the nucleic acid sequences in the genomic acid fragments. The hybridization step is preferably performed in a solution under standard conditions. For instance, the oligonucleotide probe and the genomic nucleic acid fragments should be denatured at elevated temperatures and then incubated at approximately 65° C. for 24-48 hours in the presence of carrier molecules such as yeast tRNA.

A “target molecule” as used herein is a molecule which can be covalently attached to an oligonucleotide and which has a known binding partner. Preferably the target molecule is selected from a group consisting of biotin, avidin, FITC, anti-FITC, antigen, and antibodies. A binding partner is a molecule which interacts with a target molecule. The interaction is strong enough such that the binding partner can be used to remove target from a solution. Many binding partner/target pairs are well known in the art, e.g., biotin/avidin, FITC/anti-FITC.

The target molecule is caused to interact with a binding partner. The binding partner is removed from the mixture along with any binding partner/target conjugates which have formed. The remaining genomic nucleic acid fragments are then purified and labeled and can be used in ISH methods. The hybridization and selective removal steps can be repeated several times to assure removal of all complementary nucleic acid sequences. The labeling step may be performed by any method known in the art. The nucleic acid fragments may be labeled, for instance, using chemical modification by substituting derivitized bases, by forming adducts, which can be detected by immunochemical stains, or the addition of extrinsic labels such as radioactivity and fluorescence molecules.

The binding partner is preferably immobilized on a solid support. A solid support as used herein refers to any solid material to which a binding partner can be immobilized. Solid supports, for example, include but are not limited to membranes, e.g., nitrocellulose or nylon, a bead, e.g., a magnetic bead or particle, or polymers such as polystyrene. For instance, the binding partner may be immobilized on a bead and the binding partner/target conjugate can be separated by chromatography.

The invention in another aspect is a library of nucleic acid probes for use in ISH methods. The library of nucleic acid probes is a heterogeneous mixture of labeled nucleic acid probes that are substantially complementary to unique nucleic acid fragments and are substantially free of repetitive nucleic acid sequences, and which are produced by the process of obtaining genomic nucleic acid fragments, amplifying the genomic nucleic acid fragments, hybridizing a chemically modified oligonucleotide probe to a complementary nucleic acid sequence in the genomic nucleic acid fragments, wherein the chemically modified oligonucleotide probe is an oligonucleotide associated with a target molecule, selectively removing the chemically modified oligonucleotide probe in complementary nucleic acid by selectively contacting the target molecule with a binding partner to produce a binding partner/target conjugate and separating the binding partner and binding partner/target conjugates from the genomic nucleic acid fragments, and labeling the genomic nucleic acid fragments with a label. A library as used herein refers to a plurality of nucleic acid molecules.

A “heterogenous mixture of labeled nucleic acid probes” as used herein is a plurality of nucleic acid fragments which are complementary to unique nucleic acid sequences. A “unique nucleic acid sequence” as used herein is a nucleic acid sequence of at least 12 nucleotides in length and which is present only once within an entire genome. A complementary nucleic acid is one which is capable of base pairing with a nucleic acid under stringent conditions. Hybridization conditions are described, for instance, in Sambrook, et al., Molecular Cloning: A Laboratory Manual, second edition, V 1-3, Cold Springs Harbor Laboratory Press: Cold Spring Harbor, N.Y., 1989. The preferred length of the probe is at least 15 nucleotides. The number of nucleic acid fragments within the mixture may vary widely depending upon their use. For instance, if the mixture is used as a probe for ISH to identify the presence of certain specific nucleic acid sequences within a subregion of a chromosome, then the number of nucleic acid fragments will be less than if the ISH is performed to identify nucleic acids in a plurality of chromosomes. The number of fragments will also depend upon various experimental conditions such as the unit length of nucleic acid per unit volume that can be maintained in solution. Such parameters are well known to those of ordinary skill in the art.

The heterogenous mixture of labeled nucleic acid probes is substantially free of repetitive nucleic acid sequences. “Substantially free” as used herein refers to elimination of at least 95% of known repetitive sequences.

The step of obtaining genomic nucleic acid fragments may be performed by many methods known in the art. Preferably, the genomic nucleic acid fragments are obtained from isolated chromosome specific DNA. If the heterogeneous mixture of labeled nucleic acid probes are made from a single chromosome, then that chromosome is isolated and DNA is extracted from the isolated chromosomes. Individual chromosomes can be isolated by a variety of methods known in the art. One method involves the isolation of human chromosomes from hybrid cell lines formed between human cells and non-human cells. As these hybrid cells are propagated many of the human chromosomes are lost and thus cells can be formed which contain a single human chromosome. Another method for isolating chromosomes is by direct flow sorting of metaphase cells. Such methods can be accomplished using commercially available equipment such as fluorescence-activated sorting instruments, e.g., Becton, Dickinson & FACS-II. DNA is then extracted from the isolated chromosomes by routine methods known in the art, e.g., Sambrook, et al., Molecular Cloning: A Laboratory Manual, second edition, V 1-3, Cold Springs Harbor Laboratory Press: Cold Spring Harbor, N.Y., 1989.

A preferred means for isolating individual chromosomes and regions thereof is through the use of yeast artificial chromosomes (YACs), bacterial artificial chromosomes (BACs), or P1-derived artificial chromosomes (PACs) containing specific human chromosomes. Many YAC and BAC clones are well known in the art and specific regions of chromosomes within these YAC, BAC, and PAC clones can be selected based on published maps as well as data obtained from websites such as the Whitehead Institute Center for genome research and CEPH-Genethon websites (http:\\www.genome.wi.mit.edu and http:\\www.cephb.fr, respectively). The BAC, YAC, and PAC DNA inserts can be isolated, and separated from their respective chromosomes, by methods that are well known in the art.

In preferred embodiments the YAC clone inserts can be isolated from yeast chromosomes by pulsed field gel electrophoresis, and specific size fractions may then be removed from the gels and purified. Pulsed field gel electrophoresis (PFGE) involves the resolution of large sized DNA molecules by periodically changing the electric field pattern during electrophoresis. The changes in field pattern reorient the DNA molecules and the separating medium, thus improving DNA separation. PFGE techniques have been extensively described in the prior art, such as U.S. Pat. No. 5,135,628, which is incorporated by reference. The use of PFGE to isolate the YAC clone inserts from the yeast chromosomes provides unexpectedly good results. The DNA, in yeast cells containing YACs, is about 97% yeast chromosomal DNA (i.e. the normal yeast chromosomes) whereas 3% is the YAC. The YACs contain 1 Megabase-length human inserts (one million base pairs of DNA) whereas the yeast chromosomal DNA is about 30 megabases in total length. It was found according to the invention that when DNA is isolated from the yeast using PFGE the 1 Mb YAC is separated from similarly-sized individual yeast chromosomes. It was discovered that 500 ng of non-PFGE purified, subtracted, YAC probe is required to perform FISH analysis on one specimen but that only 20 ng of PFGE-purified, subtracted, YAC probe is required to do FISH on one specimen. Thus the PFGE-purified material is 25-fold more potent because the irrelevant yeast chromosomal “filler” DNA sequences have been eliminated and only the actual human insert (probe) remains.

Once isolated, the human chromosomal DNA can be amplified. The term “amplification” as used herein refers to a method of producing complementary nucleic acid, such as PCR and other well-known methods. The step of amplifying the genomic nucleic acid fragments is preferably carried out using PCR. For instance, the nucleic acid fragments can be blunt ended and ligated to an adapter, such as the T-1/T-2 adapter, and then amplified using PCR. The amplified fragments can then be purified using techniques known in the art or commercially available kits such as QIA-quick PCR purification kits (QIAGEN).

Once the chromosomal DNA is prepared the subtractive hybridization methods of the invention can be used to synthesize ISH probes. Subtractive hybridization has been used in the prior art primarily for analysis of differences between two DNA populations.⁴⁷⁻⁵¹ Subtraction enables enrichment of the population of interest, (also referred to as tracer DNA), for sequences absent or substantially under represented in the nucleic acid population (also referred to as the driver). The methods according to the invention preferably utilize subtraction to remove repetitive sequences, while preserving unique sequences, in the mega-YAC contigs. The physical basis of the subtraction lies in the kinetics of DNA annealing in solution.⁵⁰ A molar excess of biotinylated repetitive DNA is used causing the denatured repetitive sequences in the nucleic acid sample population are far more likely to anneal to their counterparts in the biotinylated population rather than to each other. Biotinylated molecules, including binding partner/target hybrids, are subsequently removed from solution using a matrix of avidin-conjugated beads. The method maximizes both quantitative removal of repetitive sequences and retention of sequence complexity in the tracer.

We demonstrate in the Examples below that subtracted mega-YAC probes may be amplified through at least four rounds of PCR without diminishing the ISH signal intensity. Each 25-cycle round of PCR amplifies the starting material 500- to 1000-fold. Additional amplification is then achieved using random ocismer priming⁴⁵ or PCR to incorporate labeled nucleotides. Hence, subtraction converts 250 ng of adapter-ligated tracer DNA into a virtually permanent resource that can serve to generate kilogram amounts of repeat-sequence-depleted probes for ISH. This approach is particularly advantageous in the case of CEPH mega-YACs, which are known to be unstable in the strain of yeast in which they were cloned.⁵⁷⁻⁵⁸ CEPH mega-YACs often develop large deletions due to recombination and selection events. By converting contigs of mega-YAC clones into adapter-ligated libraries, we have created stable and readily available probes.

There are several well established techniques for production of large-insert ISH probes. These include Alu-PCR and DOP-PCR, which are rapid and universally applicable methods for amplification of YAC or BAC human inserts.⁵⁰⁻⁶¹ Alu-PCR is performed using Alu-sequence primers. An advantage in this approach is that Alu-containing, ie., human, sequences are amplified. However, the amplified sequences are limited primarily to those situated between closely neighboring, and appropriately oriented. Alu repeats, thus biasing the final pool of amplified DNA fragments, DOP-PCR is performed using degenerate oligonucleotide primers. This method, although nonselective for human versus bacterial or yeast sequences, may enable a more complex and representative amplification of the human insert than does Alu-PCR. Both Alu-PCR and DOP-PCR are effective and straight-forward methods requiring substantially less up-front effort than subtraction. However, neither approach is designed to generate probes depleted of repetitive sequences. In our experience, Alu-PCR and DOP-PCR YAC probes are generally associated with a lower signal-to-noise ratio, despite Cot-1 pre-annealing, than subtracted probes. DOP-PCR chromosome painting probes, on the other hand, have been extremely successful.⁵⁴⁻⁶²

The EWS and MYC probe sets described in the Examples are particularly effective in a screening mode. EWS translocations are found in Ewing's sarcoma, clear-cell sarcoma, desmoplastic small-round-cell tumor, extraskeletal myxoid chondrosarcoma, and (infrequently) myxoid liposarcoma. At least nine different partner genes participate in the translocation-related EWS fusion oncogenes in these tumors.²⁶⁻³⁴ The EWS ISH screening approach allows efficient evaluation of an EWS rearrangement in any of the aforementioned tumors. In cases with abnormal ISH patterns, the specific fusion oncogene can then be established using appropriate oligonucleotide primers, by reverse transcription PCR. This screening approach also enables localization of previously uncharacterized translocation partners. MYC translocations are also particularly amenable to ISH detection. Translocation breakpoints, in HIV-associated and endemic Burkitt lymphomas, are often 100 to 300 kb upstream of MYC.⁶⁴⁻⁶⁵ whereas translocation breakpoints in most sporadic Burkitt lymphomas involve MYC exon 1 or intron 1.^{64,65} However, 10% to 20% of sporadic Burkitt lymphomas contain arrangements of κ or λ light chain loci, and these cases typically have translocation breakpoints 200 to 300 kb downstream of MYC.⁶⁶ A MYC ISH probe set with a 500-kb gap on either side of the gene (FIG. 1) was designed according to the invention such that virtually all MYC translocations, whether upstream, intragenic, or downstream, are detected.

“ISH” as used herein is a method for detecting and localizing nucleic acids within a cell or tissue preparation. The method provides both quantitative and spacial information concerning the nucleic acid sequences within an individual cell or chromosome. ISH has been commonly been used in the areas of prenatal genetic disorder diagnosis, molecular cytogenetics, to detect gene expression and overexpression, to identify sites of gene expression, to map genes, to localize target genes and to identify various viral and microbial infections, tumor diagnosis in vitro fertilization analysis, analysis of bone marrow transplantation and chromosome analysis. The technique involves the use of labeled nucleic acid probes which are hybridized to a chromosome or mRNA in cells that are immobilized on a slide. The probes can be labeled with fluorescent molecules, in a procedure known as fluorescent In situ hybridization (FISH), see, e.g., Kuo et al., Am. J. Hum. Genet., v. 49, p. 112-119, 1991). An example of ISH and FISH methods is provided in U.S. Pat. No. 5,750,340, issued to Kim et al.

ISH methods involve the fixation of tissue or biological samples on a surface, prehybridization treatment to increase the accessability of target DNA in the sample and to reduce non-specific binding, hybridization of the labeled library of nucleic acid probes to the DNA, post-hybridization washes to remove unbound probe, and detection of the hybridized probes. Each of these steps is well known in the art and has been performed under many different experimental conditions. Set forth below are some commonly used methods for performing ISH. The materials of the invention and the genomic subtractive hybridization steps of the invention can be used with any ISH procedure.

The tissue or biological sample can be fixed to a surface using fixatives. Preferred fixatives cause fixation of the cellular constituents through a precipitating action which is reversible, maintains a cellular morphology with the nucleic acids in the appropriate cellular location and does not interfere with nucleic acid hybridization. Fixatives include, for example, but are not limited to formaldehyde, alcohols, salt solutions, mercuric chloride, sodium chloride, sodium sulfate, potassium dichromate, potassium phosphate, ammonium bromide, calcium chloride, sodium acetate, lithium chloride, cesium acetate, calcium or magnesium acetate, potassium nitrate, potassium dichromate, sodium chromate, potassium iodide, sodium iodate, sodium thiosulfate, picric acid, acetic acid, sodium hydroxide, acetones, chloroform, glycerin, and thymol.

After being fixed on a surface the samples are treated to remove proteins and other cellular material which may cause nonspecific background binding. Agents which remove protein include enzymes such as pronase or proteinase K, or mild acids, such 0.02-0.2N HCl. RNA can be removed with RNase.

The DNA on the surface must then be denatured so that the oligonucleotide probes can bind to give a signal. Denaturation can be accomplished by varying the pH, increasing temperature, or organic solvents such as formamide. The labeled probe is then hybridized with the denatured DNA under standard hybridization conditions.

The tissue or biological sample is any material which is composed of or contains cells or portions of cells. The cells may be living or dead and should contain substantially intact membranes sufficient to preserve the nucleic acid within the cell. The material may be deposited on the solid support using standard techniques such as sectioning of tissue or smearing or cytocentrifugation of single cell suspensions. Many types of solid supports may be used, including but not limited to, glass, nitrocellulose, scotch tape, nylon, or gene screen plus. A preferred support is a glass microscope slide.

A label as used herein is any molecule which may be detected. For instance, labels include but are not limited to ³²P, ¹⁴C, ¹²⁵I, ³H, ³⁵S biotin, avidin, fluorescent or enzymatic molecules. The nucleic acid may be labeled with biotin and then detected with avidin conjugated to a fluorescent, enzymatic, or colloidal gold conjugate. Biotin labeled nucleotides s can be incorporated into the nucleic acid by nick translation, enzymatic, or chemical means.

In another aspect the invention is a probe for genomic subtractive hybridization. The probes of the mixture are isolated chemically modified oligonucleotide probes, wherein the chemically modified oligonucleotide probes are oligonucleotides associated with a target molecule selected from the group consisting of biotin, avidin, FITC, anti-FITC, antigen, and antibodies and wherein the oligonucleotide probes are a mixture of oligonucleotides complementary to repetitive nucleic acid sequences and yeast nucleic acid sequences.

An “isolated chemically modified oligonucleotide probe” is a chemically modified oligonucleotide probe as described above which has been substantially separated and purified away from nucleic acid sequences in the cell of the organ in which the nucleic acid naturally occurs. The term isolated, thus, encompasses a chemically modified oligonucleotide probe wherein the oligonucleotide probe has a repetitive nucleic acid sequence and yeast DNA which is in a composition that is free of other oligonucleotides, including other unique oligonucleotides. The isolated chemically modified oligonucleotide probes, as used herein, encompasses a set of oligonucleotides having a mixture of repetitive nucleic acid sequences and yeast DNA, but does not encompass oligonucleotides having unique sequences.

### EXAMPLES

### Example 1

### Preparation of Chromosomes

Selection of YACs for MYC and EWS Translocation Detection

The MYC gene maps to chromosome subband 8q24.1 and is involved in three well characterized translocations in Burkitt lymphomas. The EWS gene maps to chromosome band 22q12 and is involved in at least eight different translocations in soft-tissue tumors. YAC clones centromeric and telomeric to these genes were selected based on published maps that were cross-referenced with physical mapping data from the Whitehead Institute Center for Genome Research and CEPH-Généthon websites (http://www.genome.wi.mit.edu and http://www.cephb.fr., respectively). YAC clones were selected based on appropriate map location and absence of features suggesting chimerism. Potential chimerism was determined using both sequence-lagged site (STS) and Alu-PCR data from the Whitehead Institute and CEPH. All YAC clones were obtained from Research Genetics (Huntsville, Ala.), and YAC DNAs were isolated as described previously. Chimerism was evaluated formally by fluorescence ISH (FISH) against normal male lymphocyte metaphase and interphase preparations and chimeric clones were excluded from the final contigs. FIG. 1 depicts the YAC clones that comprise the centromeric and telomeric contigs flanking MYC and EWS on chromosomes 8 and 22.

### Example 2

### Preparation of Nucleic Acid Sample from Chromosomal DNA

The nucleic acid sample (referred to herein as Tracer DNA) which is used to generate a library of probes for ISH discussed below was prepared by combining 2 μg of each pulsed field gel purified clone from a particular contig. These pooled DNAs were then sonicated to 0.1 to 8 kb and size-fractionated on 1.5% agarose gels. The 0.4- to 2-kb fractions were cut from the gels, purified using QlAquick gel extraction kits (QIAGEN, Santa Clarita, Calif.) blunt ended and ligated to the T-1/T-2 adapter. The T-1/T-2 adapter was constructed by annealing polyacrylamide gel electrophoresis (PAGE)-purified oligos 5′CTGAGCGGAATTCGTGAGACC (T-1) SEQ. ID. NO: 1 and 5′GGTCTCACGAATTCCGCTCAGTT (T-2) SEQ. ID. NO: 2. Adapter-ligated fragments were then PCR amplified, in multiple 25 μl reactions, using the T-1 sequence as primer. Amplified fragments were purified using QIA-quick PCR purification kits (QIAGEN) and then eluted in 1 mmol/L Tris/Cl, pH 6.0. PCR reactions here and elsewhere, unless otherwise indicated, were done in 25 μl volumes using KlenTaq reaction buffer (Clontech, Palo Alto, Calif.). 0.2 mmol/L dNTPα, 1.2 μmol/L PAGE-purified primer, and 0.1 U/ml KlenTaq DNA polymerase (Clontech). PCR cycling conditions were 94° C. for 30 seconds, 55° C. for 30 seconds, and 72° C. for 3 minutes for 25 to 30 cycles, followed by 72° C. for 9 minutes.

### Example 3

### Preparation of Chemically Modified Oligonucleotide Probe

The chemically modified oligonucleotide probes in the form of biotinylated repetitive oligonucleotide probes (referred to herein as Driver DNA) were prepared from three nonchimeric chromosome 21 YAC clones (746-b-10, 745-c-11, 615-c-9) that are known to be rich in repetitive sequences or from Cot-1 DNA which is enriched for repetitive DNA sequences. YAC DNA was isolated, as described, from a 500 ml culture consisting of all three clones. Nucleic acid thus isolated was precipitated twice in 6.5% polyethylene glycol/0.8 mol/L sodium chloride, washed in 70% ethanol, and dissolved in distilled water. Twenty micrograms of this DNA was sonicated and size fractionated as described above. Fragments ranging in size from 0.4 and 2 kb were gel purified, blunt ended, and ligated to a D-40/D-41 adapter constructed by annealing PAGE-purified oligos 5′AATTCTTGCGCCTTAAACCAAC (D-40) SEQ. ID. NO: 3 and 5′GTTGGTTTAAGGCGCAAG (D-41) SEQ. ID. NO: 4. PCR was performed using 5′(biotin)AATTCTTGCGCCTTAAACCAAC (D-40B) SEQ. ID. NO: 5 as primer. A second round of PCR was performed using the first-round product as the template. In the second round of PCR, the concentration of D-40B was increased to 6 mmol/L, the DNTP concentration was increased to 0.4 mmol/L, and the number of cycles was reduced to 20. Several hundred micrograms of biotinylated YAC repetitive oligonucleotide probes were generated in multiple 25 ml reactions. The biotinylated PCR products were purified using Qiaquick PCR purification kits (QIAGEN), precipitated in ethanol, and dissolved at 1.5 mg/ml in EE buffer (10 mmol/L 2hydroxyethyl]piperazine-N′-[3-propanesulfonic acid (NaEPPS), 1 mmol/L EDTA, pH 8.0).

### Example 4

### Subtractive Hybridization

Genomic subtractive hybridization removes sequences from a tracer DNA population by hybridizing with a molar excess of driver DNA. The driver DNA is chemically modified, e.g., with a biotin, such that it may be selectively removed from solution along with driver-tracer hybrid molecules. Briefly, MYC and EWS-region YAC contigs (FIG. 1) were repeatedly hybridized with a 40-fold excess of biotinylated YACs containing abundant repetitive, e.g., Alu and LINE element sequences. Consequently, repetitive sequences presenting the EWS- and MYC-region contigs were quantitatively removed. The detailed methods are set forth below.

Subtraction was performed by mixing 250 ng of tracer DNA with 10 μg of biotinylated driver DNA, 2 μg of T-1, 5 μg of yeast tRNA as carrier. This mixture was denatured at 99° C. for 1 minute, lyophilized, redissolved in 5 μl of EE buffer/1 mol/L NaCl, and then incubated at 65° C. for 24 to 48 hours. Biotinylated molecules (including tracer-driver hybrids) were removed using avidin-polystyrene beads as described. Remaining unbiotinylated tracer fragments were precipitated in ethanol before proceeding with the next round of subtraction. Each of three rounds of subtraction was performed exactly as described above. After the third round, remaining tracer fragments were amplified by PCR using the T-1 sequence as primer.

### Example 5

### In Situ Hybridization

Probe Preparation

The subtracted contigs telomeric to the EWS and MYC loci (EWS.T and MYC.T, respectively, FIG. 1) were labeled with biotin using the BioPrime random octamer priming kit (Gibco BRL/Life Technologies, Gaithersburg, Md.). The subtracted contigs centromeric to the EWS and MYC loci (EWS.C and MYC.C, respectively, FIG. 1) were labeled with fluorescein isothiocyanate (FITC), also by random octamer priming. The final nucleotide concentrations for FITC labeling were 0.2 mmol/L dCTP, 0.2 mmol/L dGTP, 0.2 mmol/L DATP, 0.1 mmol/L dTTP, and 0.1 mmol/L FITC-12-dUTP (NEN, Boston, Mass.). Residual primers and unincorporated nucleotides were removed by S-200H1R spin-column chromatography (Pharmacia, Uppsala, Sweden). The purified products were precipitated in ethanol and dissolved in a solution containing 50% formamide, 10% dextran sulfate, and 2× SSC (0.3 mol/l sodium chloride, 0.03 mol/L sodium citrate, pH 7.0).

Slide Preparation

Formalin-fixed, paraffin-embedded 4-μm tissue sections were applied to silanized slides, baked at 65° C. for 16 hours, and stored at room temperature. Slides were processed for ISH using Oncor Tissue kits (Oncor, Gaithersburg, Md.) according to the manufacturer's specifications with minor variations. Briefly, after deparaffination in xylene and dehydration in 100% ethanol, all tissue sections were incubated in 30% pretreatment solution for 15 minutes, followed by 15 to 40 minutes of protease treatment. Digestion times were optimized on a case-by-case basis. Alternately, paraffin sections were prepared for ISH using a combination of microwaving at 92° C. followed by protein digestion using pepsin. Slides were denatured in a solution containing 70% formamide, 2× SSC, pH 7.0, at 75° C. for 8 minutes. Slides were dehydrated in ice-cold 70%, 85%, and 95% ethanol and then air dried. Cytogenetic preparations were processed according to standard methods.

Hybridization and Detection

Aliquots of labeled DNA were diluted to a concentration of 100 to 200 ng/μl in hybridization solution (50% formamide, 10% dextran sulfate, 2× SSC), denatured at 75° C. for 5 minutes, placed immediately onto denatured slides, and covered with a glass coverslip that was sealed with rubber cement. The slides were then placed in a humidified chamber at 37° C. for 12 to 16 hours. Slides were washed in 0.5× SSC at 72° C. for 5 minutes and then in PN buffer (0.1 mol/L sodium phosphate, pH 8.0, 0.1% Nonidet P-40) at room temperature. Biotinylated probes were detected with μg/ml Texas Red-strepavidin (Zymed Laboratories, South San Francisco, Calif.). FITC-labeled probes were visualized directly and, in some cases, amplified using rabbit anti-FITC and FITC anti-rabbit (Zymed). For calorimetric detection, sequential peroxidase reactions were performed using horseradish peroxidase (HRP)-conjugated goat anti-FITC (Zymed Laboratories, South San Francisco, Calif.) with the diaminobanzidine (DAB) substrate kit (Zymed), followed by HRP-conjugated strapavidin (Zymed) with the VIP substrate kit (Vector). Cells were counterstained with Gill's hematoxylin (Vector) and mounted in Permount (Sigma-Aldrich Corp., St. Louis, Mo.).

Dot Blotting

Total genomic DNA from Saccharomyces cerevisae strain AB1380 (a negative control) as well as subtracted and unsubtracted tracer DNA (EWS.C and EWS.T) were evaluated. Amounts of 300, 100, 30, 10, and 1 ng of each DNA were denatured and spotted onto a positively charged nylon membrane, which was then baked. The membrane was probed successively with radiolabeled human Cot-1 DNA (Gibco BRL/Life Technologies) and total yeast genomic DNA (strain AB1380).

Subtraction of Human and Yeast Repetitive Sequences

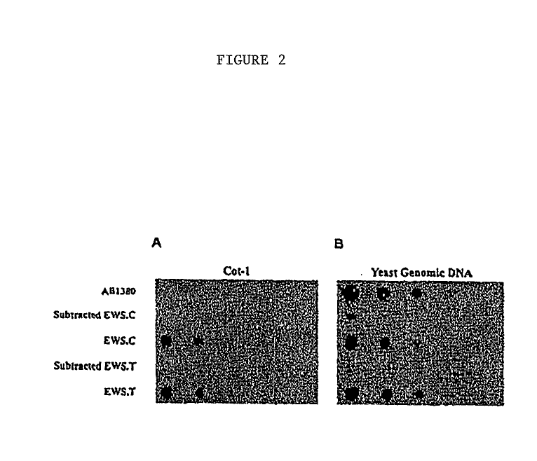

Dot blots of subtracted and unsubtracted tracer DNAs were evaluated for the presence of human repetitive sequences using human Cot-1 (repetitive-sequence-enriched) DNA as probe. This experiment demonstrated complete subtraction of human repetitive sequences from the tracer DNAs (FIG. 2A). Reprobing with total yeast genomic DNA demonstrated substantial removal of yeast sequences (FIG. 2B). All subtracted tracer DNA libraries were then evaluated as FISH probes, in the absence of Cot-1 competitor DNA or pre-annealing. FISH signals localized exclusively to the expected chromosome regions, and signal intensities were identical to those obtained using unsubtracted DNAs (the latter preannealed and hybridized in the presence of excess Cot-1 DNA to suppress nonspecific background staining). Potential PCR-related biases were evaluated by subjecting the subtracted probe pools to four successive rounds of PCR amplification. Probe aliquots were labeled after each round of amplification and were hybridized against two different slides. No dimunition in probe signal intensity was seen after four rounds of amplification. This experiment was validated by repeating the four rounds of PCR amplification using another aliquot of subtracted probe. Again, there was no fall-off FISH signal intensity after the fourth round of PCR. The Gibco BioPrime random octamer labeling approach permitted substantial amplification of the template DNA during the labeling process. Typical yields, starting with 200 ng of subtracted tracer, were 5-10 μg (25-60 fold amplification) of labeled DNA after a 5 hour, 50 μl random priming reaction. PCR incorporation, using the T-1 adapter primers with biotin- or digoxigenin-conjugated nucleotides, was an equally effective alternative to random priming.

Application 1: Evaluation of EWS-Region Rearrangements

The subtracted EWS-region ISH probe set was evaluated against 1) a primary cutaneous Ewing's sarcoma, 2) a clear-cell sarcoma, and 3) a tibial Ewing's sarcoma that lacked, cytogenetically, a typical (11:22). The cutaneous Ewing's sarcoma as reported previously, was an axillary lesion in a 19-year old woman. The clear-cell sarcoma was a right knee mass in a 30-year-old woman. The clear-cell sarcoma was a right knee mass in a 39-year-old woman. The histological differential diagnosis, for the knee mass, included both clear-cell sarcoma (melanoma of soft parts) and metastatic cutaneous melanoma. Greater than 65% of clear-cell carcomas contain a (12:22)(q13.q12), resulting in fusion of EWS and ATF-1²⁷ whereas this translocation has not been reported in cutaneous malignant melanoma. FISH analysis of 4 μm paraffin sections revealed splitting of one EWS.C/EWS.T probe pair, consistent with EWS-region rearrangement, in both the cutaneous Ewing's sarcoma and putative clear-cell sarcoma. The tibial Ewing's sarcoma, diagnosed in a 17-year-old boy, had a cytogenetically aberrant chromosome 22 homolog, whereas chromosomes 2, 7, 11, 17, and 21 (chromosomes containing ETS family genes involved in known Ewing's sarcoma EWS fusions) were unremarkable by banding analysis. EWS.C/EWS.T FISH evaluation revealed a reciprocal translocation (1:22)(q42:q12), of the EWS region, and reverse transcriptase PCR was negative for EWS-FLI1 or EWS-ERG fusion transcripts. These data support a unique EWS translocation, potentially involving a novel ETS family locus on chromosome band 1q42.

Application 2: Evaluation of MYC-Region Rearrangement

The subtracted MYC-region ISH probe was evaluated against a malignant pleural effusion in which cytological evaluation, but not immunophenotype, was classical for Burkill's lymphoma. The pleural fluid was from a 10-year-old boy with a 2-month history of anorexia and abdominal pain and with radiolocal evidence of mesenteric/mediastinal adenopathy and bilateral pleural effusions. Cytological evaluation revealed a homogeneous population of small lymphoid cells with bluish vacuolated cytoplasm and round nongrooved nuclei, whereas flow immunophenotyping was notable for the absence of surface immunoglobulin. Cytogenetic banding studies were inconclusive because the chromosome morphology was poor. FISH analysis, using the subtracted MYC.C/MYC.T probe set demonstrated MYC-region rearrangement in metaphase and interphase cells. MYC-region rearrangement was also demonstrated convincingly by calorimetric detection.

In summary, we report a new method for constructing and synthesizing DNA probes from multimegabase YAC contigs. This method enables creation of adapter-ligated DNA libraries that are free of certain sequences such as repetitive sequences. We demonstrate that subtracted unique-sequence probes are detected readily using standard fluorescence and calorimetric reagents. The probes are labeled conveniently and are hybridized without competitor DNA or pre-annealing, thus simplifying the In situ hybridization protocol.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, patents and patent publications that are recited in this application are incorporated in their entirety herein by reference.

### REFERENCES

1. Rabbitts T H: Chromosomal translocations in human cancer. Nature 1994, 372:143-149

2. Mitelman F, Mertens F, Johansson B: A breakpoint map of recurrent chromosomal rearrangements in human neoplasia. Nat Genet 1997, 15 Spec No:417-474

3. Look A T: Oncogenic transcription factors in the human acute leukemias. Science 1997, 278:1059-1064

4. Delattre O, Zucman J, Melot T, Garau X S, Zucker J M, Lenoir G M, Ambros P F, Sheer D, Turc-Carel C, Triche T J, Aurias A, Thomas G: The Ewing family of tumors—a subgroup of small-round-cell tumors defined by specific chimeric transcripts. N Engl J Med 1994, 331:294-299

5. Sandberg A A, Turc-Carel C, Gemmill R M: Chromosomes in solid tumors and beyond. Cancer Res 1988, 48:1049-1059

6. Sreekantaiah C, Ladanyi M, Rodriguez E, Chaganti R S: Chromosomal aberrations in soft tissue tumors. Relevance to diagnosis, classification, and molecular mechanisms. Am J Pathol 1994, 144:1121-1134

7. Speel E J, Ramaekers F C, Hopman A H: Cytochemical detection systems for in situ hybridization, and the combination with immunocytochemistry, ‘who is still afraid of red, green and blue?’. Histochem J 1995, 27:833-858

8. Hopman A H, Claessen S, Speel E J: Multi-colour brightfield in situ hybridisation on tissue sections. Histochem Cell Biol 1997, 108:291-298

9. Hopman A H, Poddighe P J, Smeets A W, Moesker O, Beck J L, Vooijs G P, Ramaekers F C: Detection of numerical chromosome aberrations in bladder cancer by in situ hybridization. Am J Pathol 1989, 135:1105-1117

10. Cremer T, Lichter P, Borden J, Ward D C, Manuelidis L: Detection of chromosome aberrations in metaphase and interphase tumor cells by in situ hybridization using chromosome-specific library probes. Hum Genet 1988, 80:235-246

11. Pinkel D, Landegent J, Collins C, Fuscoe J, Segraves R, Lucas J, Gray J: Fluorescence in situ hybridization with human chromosome-specific libraries: detection of trisomy 21 and translocations of chromosome 4. Proc Natl Acad Sci U S A 1988, 85:9138-9142

12. Tkachuk D C, Westbrook C A, Andreeff M, Donlon T A, Cleary M L, Suryanarayan K, Homge M, Redner A, Gray J, Pinkel D: Detection of bcr-abl fusion in chronic myelogeneous leukemia by in situ hybridization. Science 1990, 250:559-562

13. Xiao S, Renshaw A A, Cibas E S, Hudson T J, Fletcher J A: Novel fluorescence in situ hybridization approaches in solid tumors: Characterization of frozen specimens, touch preparations, and cytologic preparations. Am J Pathol 1995, 147:896-904

14. Poddighe P J, Moesker O, Smeets D, Awwad B H, Ramaekers F C, Hopman A H: Interphase cytogenetics of hematological cancer: comparison of classical karyotyping and in situ hybridization using a panel of eleven chromosome specific DNA probes. Cancer Res 1991, 51:1959-1967

15. Gray J W, Pinkel D: Molecular cytogenetics in human cancer diagnosis. Cancer 1992, 69:1536-1542

16. Wolman S R: Fluorescence in situ hybridization: a new tool for the pathologist. Hum Pathol 1994, 25:586-590

17. Tiainen M, Hopman A, Moesker O, Ramaekers F, Wessman M, Laasonen A, Pyrhonen S, Tammilehto L, Mattson K, Knuutila S: Interphase cytogenetics on paraffin sections of malignant pleural mesothelioma. A comparison to conventional karyotyping and flow cytometric studies. Cancer Genet Cytogenet 1992, 62:171-179

18. Manuelidis L, Langer-Safer P R, Ward D C: High-resolution mapping of satellite DNA using biotin-labeled DNA probes. J Cell Biol 1982, 95:619-625

19. Pinkel D, Straume T, Gray J W: Cytogenetic analysis using quantitative, high-sensitivity, fluorescence hybridization. Proc Natl Acad Sci U S A 1986, 83:2934-2938

20. Lichter P, Tang C J, Call K, Hermanson G, Evans G A, Housman D, Ward D C: High-resolution mapping of human chromosome 11 by in situ hybridization with cosmid clones. Science 1990, 247:64-69

21. Kallioniemi A, Kallioniemi O P, Sudar D, Rutovitz D, Gray J W, Waldman F, Pinkel D: Comparative genomic hybridization for molecular cytogenetic analysis of solid tumors. Science 1992, 258:818-821

22. Speicher M R, Gwyn Ballard S, Ward D C: Karyotyping human chromosomes by combinatorial multi-fluor FISH. Nat Genet 1996, 12:368-375

23. Schrock E, du Manoir S, Veldman T, Schoell B, Wienberg J, Ferguson-Smith M A, Ning Y, Ledbetter D H, Bar-Am I, Soenksen D, Garini Y, Ried T: Multicolor spectral karyotyping of human chromosomes. Science 1996, 273:494-497

24. Taub R, Kirsch I, Morton C, Lenoir G, Swan D, Tronick S, Aaronson S, Leder P: Translocation of the c-myc gene into the immunoglobulin heavy chain locus in human Burkitt lymphoma and murine plasmacytoma cells. Proc Natl Acad Sci U S A 1982, 79:7837-7841

25. Davis M, Malcolm S, Rabbitts T H: Chromosome translocation can occur on either side of the c-myc oncogene in Burkitt lymphoma cells. Nature 1984, 308:286-288

26. Delattre O, Zueman J, Plougastel B, Desmaze C, Melot T, Peter M, Kovar H, Joubert I, de Jong P, Rouleau G, Aurias A, Thomas G: Gene fusion with an ETS DNA-binding domain caused by chromosome translocation in human tumours. Nature 1992, 359:162-165

27. Zucman J, Delattre O, Desmaze C, Epstein A L, Stenman G, Speleman F, Fletcher C D, Aurias A, Thomas G: EWS and ATF-1 gene fusion induced by t(12;22) translocation in malignant melanoma of soft parts. Nat Genet 1993, 4:341-345

28. Sorensen P H, Lessnick S L, Lopez-Terrada D, Liu X F, Triche T J, Denny C T: A second Ewing's sarcoma translocation, t(21;22), fuses the EWS gene to another ETS-family transcription factor, ERG. Nat Genet 1994, 6:146-151

29. Ladanyi M, Gerald W: Fusion of the EWS and WT1 genes in the desmoplastic small round cell tumor. Cancer Res 1994, 54:2837-2840

30. Jeon I S, Davis J N, Braun B S, Sublett J E, Roussel M F, Denny C T, Shapiro D N: A variant Ewing's sarcoma translocation (7;22) fuses the EWS gene to the ETS gene ETV1. Oncogene 1995, 10:1229-1234

31. Gill S, McManus A P, Crew A J, Benjamin H, Sheer D, Gusterson B A, Pinkerton C R, Patel K, Cooper C S, Shipley J M: Fusion of the EWS gene to a DNA segment from 9q22-31 in a human myxoid chondrosarcoma. Genes Chromosom Cancer 1995, 12:307-310

32. Clark J, Benjamin H, Gill S, Sidhar S, Goodwin G, Crew J, Gusterson B A, Shipley J, Cooper C S: Fusion of the EWS gene to CHN, a member of the steroid/thyroid receptor gene superfamily, in a human myxoid chondrosarcoma. Oncogene 1996, 12:229-235

33. Panagopoulos I, Hoglund M, Mertens F, Mandahl N, Mitelman F, Aman P: Fusion of the EWS and CHOP genes in myxoid liposarcoma. Oncogene 1996, 12:489-494

34. Peter M, Couturier J, Pacquement H, Michon J, Thomas G, Magdelenat H, Delattre O: A new member of the ETS family fused to EWS in Ewing tumors. Oncogene 1997, 14:1159-1164

35. Hudson T J, Stein L D, Gerety S S, Ma J, Castle A B, Silva J, Slonim D K, Baptista R, Kruglyak L, Xu S H, Hu S, Colbert A M E, Rosenberg C, Reeve-Daly M P, Rozen S, Hui L, Wu X, Vestergaard C, Wilson K M, Bae J S, Maitra S, Gaiatsas S, Evans C A, DeAngelis M M, Ingalls K A, Nahf R W, Horton L T, Anderson M O, Collymore A J, Ye W, Kouyoumijian V, Zemsteva I S, Tam J, Devine R, Courtney D F, Renaud M T, Nguyen H, O'Connor T J, Fizames C, Faure S, Gyapay G, Dib C, Morissette J, Orlin J B, Birren B W, Goodman N, Weissenbach J, Hawkins T L, Foote S, Page DCet al.An STS-based map of the human genome. Science 1995, 270:1945-1954

36. Collins J E, Cole C G, Smink L J, Garrett C L, Leversha M A, Soderlund C A, Maslen G L, Everett L A, Rice K M, Coffey A J, Gregory S G, Gwilliam R, Dunham A, Davies A F, Hassock S, Todd C M, Lehrach H, Hulsebos T J M, Weissenbach J, Morrow B, Kucherlapati R S, Wadey R, Scarnbler P J, Kim U J, Simon M I, Peyrard M, Xie Y G, Carter N P, Durbin R, Dumanski J P, Bentley D R, Dunham I: A high-density YAC contig map of human chromosome 22. Nature 1995, 377:367-379

37. Kim U J, Shizuya H, Kang H L, Choi S S, Garrett C L, Smink L J, Birren B W, Korenberg J R, Dunham I, Simon M I: A bacterial artificial chromosome-based framework contig map of human chromosome 22q. Proc Natl Acad Sci U S A 1996, 93:6297-6301

38. Huang S F, Xiao S, Renshaw A A, Loughlin K R, Hudson T J, Fletcher J A: Fluorescence in situ hybridization evaluation of chromosome deletion patterns in prostate cancer. Am J Pathol 1996, 149:1565-1573

39. Milner J J, Cecchini E, Dominy P J: A kinetic model for subtractive hybridization. Nucleic Acids Res 1995, 23:176-187

40. Lovett M: Direct selection of cDNAs using genomic contigs. In: Current Protocols in Human Genetics. Dracopoli N C, Haines J L, Korf B R, et al, John Wiley and Sons, Inc. 1994; 6.3.1-6.3.15.

41. Xu H, Wei H, Tassone F, Graw S, Gardiner K, Weissman S M: A search for genes from the dark band regions of human chromosome 21. Genomics 1995, 27:1-8

42. Silverman G A: Purification of YAC-containing total yeast DNA. In: YAC Protocols. Markie D, ed. Totawa, N J: Humana Press Inc. 1995; 65-68.

43. Straus D, Ausubel F M: Genomic subtraction for cloning DNA corresponding to deletion mutations. Proc Natl Acad Sci U S A 1990, 87:1889-1893

44. Lanciego J L, Goede P H, Witter M P, Wouterlood F G: Use of peroxidase substrate Vector VIP for multiple staining in light microscopy. J Neurosci Methods 1997, 74:1-7

45. Mackey J, Darfler M, Nisson P, Rashtchian A: Use of random primer extension for concurrent amplification and nonradioactive labeling of nucleic acids. Anal Biochem 1993, 212:428-435

46. Hasegawa S L, Davison J M, Rutten A, Fletcher J A, Fletcher C D: Primary cutaneous Ewing's sarcoma: immunophenotypic and molecular cytogenetic evaluation of five cases. Am J Surg Pathol 1998, 22:310-318

47. Nedivi E, Hevroni D, Naot D, Israeli D, Citri Y: Numerous candidate plasticity-related genes revealed by differential cDNA cloning. Nature 1993, 363:718-722

48. Mor O, Messinger Y, Rotman G, Bar-Am I, Ravia Y, Eddy R L, Shows T B, Park J G, Gazdar A F, Shiloh Y: Novel DNA sequences at chromosome 10q26 are amplified in human gastric carcinoma cell lines: molecular cloning by competitive DNA reassociation. Nucleic Acids Res 1991, 19:117-123

49. Nussbaum R L, Lesko J G, Lewis R A, Ledbetter S A, Ledbetter D H: Isolation of anonymous DNA sequences from within a submicroscopic X chromosomal deletion in a patient with choroideremia, deafness, and mental retardation. Proc Natl Acad Sci U S A 1987, 84:6521-6525

50. Kunkel L M, Monaco A P, Middlesworth W, Ochs H D, Latt S A: Specific cloning of DNA fragments absent from the DNA of a male patient with an X chromosome deletion. Proc Natl Acad Sci U S A 1985, 82:4778-4782

51. Lamar E E, Palmer E: Y-encoded, species-specific DNA in mice: evidence that the Y chromosome exists in two polymorphic forms in inbred strains. Cell 1984, 37:171-177

52. Rosenberg M, Przybylska M, Straus D: “RFLP subtraction”: a method for making libraries of polymorphic markers. Proc Natl Acad Sci U S A 1994, 91:6113-6117

53. Reijo R, Lee T Y, Salo P, Alagappan R, Brown L G, Rosenberg M, Rozen S, Jaffe T, Straus D, Hovatta O, et al: Diverse spermatogenic defects in humans caused by Y chromosome deletions encompassing a novel RNA-binding protein gene. Nat Genet 1995, 10:383-393

54. Guan X Y, Meltzer P S, Trent J M: Rapid generation of whole chromosome painting probes (WCPs) by chromosome microdissection. Genomics 1994, 22:101-107

55. Craig J M, Kraus J, Cremer T: Removal of repetitive sequences from FISH probes using PCR-assisted affinity chromatography. Hum Genet 1997, 100:472-476

56. Lichter P, Cremer T, Borden J, Manuelidis L, Ward D C: Delineation of individual human chromosomes in metaphase and interphase cells by in situ suppression hybridization using recombinant DNA libraries. Hum Genet 1988, 80:224-234

57. Kohno K, Oshiro T, Kishine H, Wada M, Takeda H, Ihara N, Imamoto F, Kano Y, Schlessinger D: Construction and characterization of a rad51rad52 double mutant as a host for YAC libraries. Gene 1997, 188:175-181

58. Le Y, Dobson M J: Stabilization of yeast artificial chromosome clones in a rad54-3 recombination-deficient host strain. Nucleic Acids Res 1997, 25:1248-1253

59. Lengauer C, Green E D, Cremer T: Fluorescence in situ hybridization of YAC clones after Alu-PCR amplification. Genomics 1992, 13:826-828

60. Lengauer C, Riethman H C, Speicher M R, Taniwaki M, Konecki D, Green E D, Becher R, Olson M V, Cremer T: Metaphase and interphase cytogenetics with Alu-PCR-amplified yeast artificial chromosome clones containing the BCR gene and the protooncogenes c-raf-1, c-fms, and c-erbB-2. Cancer Res 1992, 52:2590-2596

61. Bohlander S K, Espinosa R, 3d, Fernald A A, Rowley J D, Le Beau M M, Diaz M O, Espinosa R: Sequence-independent amplification and labeling of yeast artificial chromosomes for fluorescence in situ hybridization. Cytogenet Cell Genet 1994, 65:108-110

62. Guan X Y, Trent J M, Meltzer P S: Generation of band-specific painting probes from a single microdissected chromosome. Hum Mol Genet 1993, 2:1117-1121

63. Joos S, Haluska F G, Falk M H, Henglein B, Hameister H, Croce C M, Bornkamm G W: Mapping chromosomal breakpoints of Burkitt's t(8;14) translocations far upstream of c-myc. Cancer Res 1992, 52:6547-6552

64. Veronese M L, Ohta M, Finan J, Nowell P C, Croce C M: Detection of myc translocations in lymphoma cells by fluorescence in situ hybridization with yeast artificial chromosomes. Blood 1995, 85:2132-2138

65. Pelicci P G, Knowles D M, 2d, Magrath I, Dalla-Favera R, Knowles D M: Chromosomal breakpoints and structural alterations of the c-myc locus differ in endemic and sporadic forms of Burkitt lymphoma. Proc Natl Acad Sci U S A 1986, 83:2984-2988

66. Zeidler R, Joos S, Delecluse H J, Klobeck G, Vuillaume M, Lenoir GM, Bornkamm G W, Lipp M: Breakpoints of Burkitt's lymphoma t(8;22) translocations map within a distance of 300 kb downstream of MYC. Genes Chromosom Cancer 1994, 9:282-287

We claim:

1. A method for genomic subtractive hybridization, comprising:
hybridizing a chemically modified oligonucleotide probe to a complementary nucleic acid sequence in a nucleic acid sample, wherein the chemically modified oligonucleotide probe is an oligonucleotide associated with a target molecule, and
selectively removing the chemically modified oligonucleotide probe and complementary nucleic acid by selectively contacting the target molecule with a binding partner to produce binding partner/target conjugates and separating the binding partner and binding partner/target conjugates from the nucleic acid sample.

2. The method of claim 1, wherein the target molecule is selected from the group consisting of avidin, biotin, FITC, anti-FITC, antigen, and antibodies.

3. The method of claim 1, wherein the oligonucleotide is a repetitive nucleic acid.

4. The method of claim 3, wherein the repetitive nucleic acid is isolated from a DNA source selected from the group consisting of YAC, BAC and PAC DNA.

5. The method of claim 3, wherein the chemically modified oligonucleotide probe is prepared by amplifying the repetitive nucleic acid using PCR and a primer attached to the target molecule.

6. The method of claim 1, wherein the nucleic acid sample is a genomic nucleic acid sample.

7. The method of claim 1, wherein the genomic nucleic acid sample is obtained from a YAC clone.

8. The method of claim 7, wherein the YAC clone is purified by pulsed field gel electrophoresis.

9. The method of claim 1, wherein the binding partner is immobilized on a support.

10. The method of claim 9, wherein the support is a bead and wherein the binding partner/target conjugates are separated from the nucleic acid sample by chromatography.

11. The method of claim 1, wherein the genomic nucleic acid sample is obtained from a DNA source selected from the group consisting of YAC, BAC and PAC DNA.

12. A library of nucleic acid probes for use in in situ hybridization methods, comprising:
a heterogenous mixture of labeled nucleic acid probes that are substantially complementary to unique nucleic acid fragments and are substantially free of repetitive nucleic acid sequences, and which are produced by the process of:
(a) obtaining genomic nucleic acid fragments;
(b) amplifying the genomic nucleic acid fragments;
(c) hybridizing a chemically modified oligonucleotide probe to a complementary nucleic acid sequence in the genomic nucleic acid fragments, wherein the chemically modified oligonucleotide probe is an oligonucleotide associated with a target molecule;
(d) selectively removing the chemically modified oligonucleotide probe and complementary nucleic acid by selectively contacting the target molecule with a binding partner and separating the binding partner and binding partner/target conjugates from the genomic nucleic acid fragments; and
(e) labeling the genomic nucleic acid fragments with a label.

13. The library of claim 12, wherein the genomic nucleic acid fragments are labeled with a fluorescent molecule.

14. The library of claim 12, wherein the heterogenous mixture of labeled nucleic acid probes are complementary to substantially all of a genomic DNA population.

15. The library of claim 12, wherein the heterogenous mixture of labeled nucleic acid probes are complementary to substantially all of a chromosome of a genomic DNA population.

16. The library of claim 12, wherein the heterogenous mixture of labeled nucleic acid probes are complementary to substantially all of a subregion of a chromosome of a genomic DNA population.

17. A method for performing in situ hybridization, comprising:
hybridizing the library of labeled probes of claim 12 to a biological sample fixed on a surface,
removing un-hybridized probe, and
detecting a signal from the hybridized probe to identify nucleic acid sequences present in the biological sample.
